(19) United States
(12) Patent Application Publication
(10) Pub. No.: US 2025/0147592 A1
(43) Pub. Date: May 8, 2025

(54) INTRACEREBRAL INFORMATION RECOGNITION DEVICE, INTRACEREBRAL INFORMATION RECOGNITION METHOD, AND STORAGE MEDIUM

(71) Applicant: JVCKENWOOD Corporation, Yokohama-shi (JP)

(72) Inventor: Takayuki Sugahara, Yokohama-shi (JP)

(21) Appl. No.: 19/017,864

(22) Filed: Jan. 13, 2025

Related U.S. Application Data
(63) Continuation of application No. PCT/JP2023/033006, filed on Sep. 11, 2023.

(30) Foreign Application Priority Data
Sep. 22, 2022 (JP) 2022-151516

(51) Int. Cl. G06F 3/01; A61B 5/16

(52) U.S. Cl. CPC G06F 3/015; A61B 5/16

(57) ABSTRACT

An intracerebral information recognition device includes: a detection unit that detects intracerebral information on a subject; and a control unit that controls a stimulation provision unit such that, in a case where unconscious thought information generated prior to a point in time of a judgment made consciously upon the subject making the judgment is included in a result of detection of the intracerebral information, a stimulation is provided at a point in time prior to the point in time of the judgment.

### CROSS-REFERENCE TO RELATED APPLICATIONS

This application is a Continuation of PCT International Application No. PCT/JP2023/033006 filed on Sep. 11, 2023 which claims the benefit of priority from Japanese Patent Application No. 2022-151516 filed on Sep. 22, 2022, the entire contents of both of which are incorporated herein by reference.

### BACKGROUND OF THE INVENTION

### 1. Field of the Invention

The present disclosure relates to an intracerebral information recognition device, an intracerebral information recognition method, and an intracerebral information recognition program.

### 2. Description of the Related Art

A technique for preventing a subject from unconsciously carrying out an action has been disclosed (see, for example, Japanese Unexamined Patent Application, Publication No. 2002-329272). In this technique, whether a hand of the subject is holding or has released an object is detected on the basis of, for example, a myoelectric potential of the subject, and a warning sound is output if a release of the object by the subject is detected.

It has been known from recent research that upon a subject making a judgment, intracerebral information related to the judgment is generated unconsciously prior to a point in time of the judgment that is made consciously. It is considered that causing an appropriate conscious recognition of such intracerebral information generated unconsciously is able to be made useful for a judgment made by a subject. Such a conscious recognition of intracerebral information unconsciously generated is not disclosed in Japanese Unexamined Patent Application, Publication No. 2002-329272.

### SUMMARY OF THE INVENTION

It is an object of the present invention to at least partially solve the problems in the conventional technology.

The above and other objects, features, advantages and technical and industrial significance of this invention will be better understood by reading the following detailed description of presently preferred embodiments of the invention, when considered in connection with the accompanying drawings.

An intracerebral information recognition device apparatus according to the present disclosure comprising:
a detection unit that detects intracerebral information on a subject;
a stimulation provision unit that provides a stimulation to the subject; and
a control unit that controls, in a case where a result of detection of the intracerebral information includes unconscious thought information generated prior to a point in time of a judgment made consciously upon the subject making the judgment, the stimulation provision unit such that the stimulation is provided at a point in time prior to the point in time of the judgment.

An intracerebral information recognition method apparatus according to the present disclosure, including:
detecting intracerebral information on a subject; and
causing, in a case where a result of detection of the intracerebral information includes unconscious thought information generated prior to a point in time of a judgment made consciously upon the subject making the judgment, the unconscious thought information to be recognized at a point in time prior to the point in time of the judgment.

A non-transitory computer-readable storage medium storing a program causing a computer apparatus according to the present disclosure to execute:
a process of detecting intracerebral information on a subject; and
a process of causing, in a case where a result of detection of the intracerebral information includes unconscious thought information generated prior to a point in time of a judgment made consciously upon the subject making the judgment, the unconscious thought information to be recognized at a point in time prior to the point in time of the judgment.

### BRIEF DESCRIPTION OF THE DRAWINGS

FIG. 1 is a schematic diagram illustrating an example of an intracerebral information recognition device according to an embodiment;

FIG. 2 is a functional block diagram illustrating an example of the intracerebral information recognition device according to the embodiment;

FIG. 3 is a diagram illustrating an example of a detection result from a detection of intracerebral information on a subject;

FIG. 4 is a flowchart illustrating an example of an intracerebral information recognition method according to the embodiment; and

FIG. 5 is a schematic diagram illustrating another example of the intracerebral information recognition device according to the embodiment.

### DETAILED DESCRIPTION OF THE PREFERRED EMBODIMENTS

An embodiment of an intracerebral information recognition device, an intracerebral information recognition method, and an intracerebral information recognition program, according to the present disclosure, will hereinafter be described on the basis of the drawings. The present invention is not to be limited by this embodiment. Furthermore, components in the following embodiment include those easily substitutable by persons skilled in the art or those that are substantially the same.

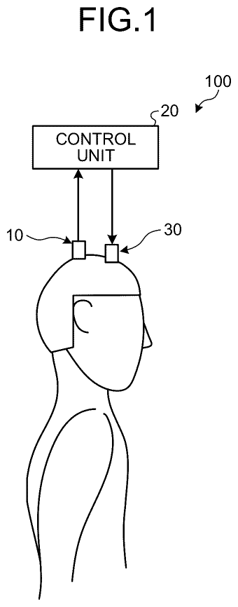

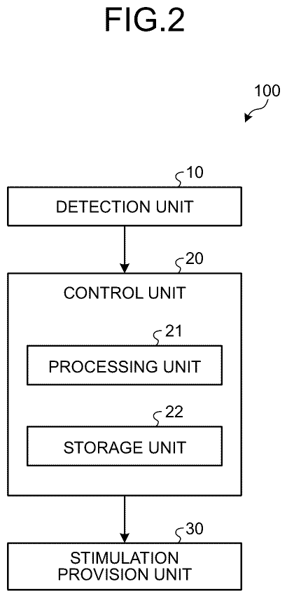

FIG. 1 is a schematic diagram illustrating an example of an intracerebral information recognition device 100 according to an embodiment. FIG. 2 is a functional block diagram illustrating the example of the intracerebral information recognition device 100 according to the embodiment. The intracerebral information recognition device 100 illustrated in FIG. 1 and FIG. 2 includes a detection unit 10, a control unit 20, and a stimulation provision unit 30.

The detection unit 10 detects intracerebral information generated prior to a point in time of a judgment made consciously upon a subject making the judgment. The detection unit 10 transmits a detection result to the control unit 20.

Examples of the intracerebral information include brain waves and intracerebral activation information. Devices that may be used to detect the intracerebral information include, for example: an electroencephalographic device; a measurement device for measurement on the basis of the principle of functional magnetic resonance imaging (fMRI) or functional near-infrared spectroscopy (fNIRS); a measurement device using invasive electrodes; and a measurement device for measurement by means of a micromachine arranged in a blood vessel of the brain.

The control unit 20 controls the stimulation provision unit 30 such that a stimulation is provided to a subject in a case where intracerebral information detected by the detection unit 10 includes unconscious thought information on the subject, the stimulation being for causing the subject to recognize the unconscious thought information before a point in time of a judgment made consciously. The unconscious thought information is information on unconscious thought of the subject.

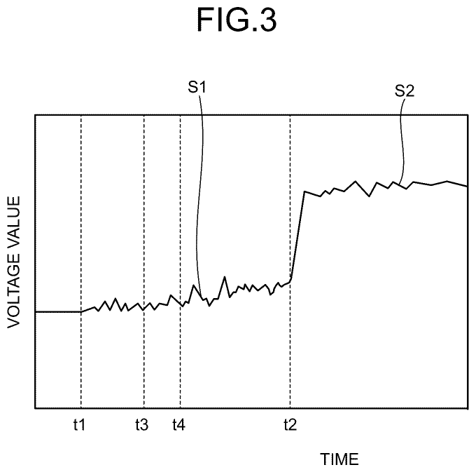

FIG. 3 is a diagram illustrating an example of a detection result from a detection of intracerebral information on a subject. FIG. 3 schematically illustrates brain waves over time upon the subject making a judgment. In FIG. 3, the vertical axis represents the value of voltage and the horizontal axis represents the time elapsed.

As illustrated in FIG. 3, a first section S1 where the value fluctuates from a point in time t1 prior to a point in time t2 of the judgment, and a second section S2 where the value fluctuates from the point in time t2 of the judgment are recognized in the brain waves of the subject. In other words, it is recognized that the intracerebral information on the subject includes: thought information prior to the point in time t2 of the judgment, that is, unconscious thought information indicating that the subject is unconsciously thinking; and thought information from the point in time t2 of the judgment, that is, information on conscious thought of the subject.

The control unit 20 has a processing unit 21 and a storage unit 22. The processing unit 21 has a processing device, such as a central processing unit (CPU), and a storage device, such as a random access memory (RAM) or a read only memory (ROM).

The processing unit 21 determines whether or not the intracerebral information detected includes unconscious thought information on the subject. For example, a pattern of unconscious thought information on the subject may be obtained beforehand and stored in the storage unit 22. In this case, the processing unit 21 is able to determine whether or not the intracerebral information detected includes unconscious thought information on the basis of information stored in the storage unit 22. That is, the unconscious thought information can also be said to be information indicating a predetermined pattern with respect to the intracerebral information.

In a case where the processing unit 21 has determined that the intracerebral information detected includes unconscious thought information, the processing unit 21 determines a form of a stimulation to be provided to the subject. The processing unit 21 determines the form of the stimulation so that a stimulation corresponding to the unconscious thought information is provided to the subject. The form of the stimulation may be that of a stimulation that the stimulation provision unit 30 is capable of providing. At a point in time of the determination of the form of the stimulation, the processing unit 21 determines whether or not that point in time is prior to a point in time of a judgment made consciously. For example, a pattern of information on conscious thought of the subject or a pattern of thought information at a point in time of a judgment by the subject may be obtained beforehand and stored in the storage unit 22. In this case, if the pattern of the information on the conscious thought or the pattern of the thought information at the point in time of the judgment is not detected, the processing unit 21 is able to determine that the point in time is prior to the point in time of the judgment. In a case where the processing unit 21 has determined that the point in time is prior to the point in time of the judgment, the processing unit 21 causes a stimulation to be provided to the subject in the form that has been determined.

The storage unit 22 stores various kinds of information. For example, the storage unit 22 has a storage, such as a hard disk drive or a solid state drive. An external storage medium, such as a removable disk, may be used as the storage unit 22. The storage unit 22 stores a program for the processing unit 21 to implement the above described functions, and data.

The stimulation provision unit 30 stimulates a sense of the subject according to the form of the stimulation determined by the processing unit 21. The stimulation provision unit 30 provides a stimulation to a predetermined region in the brain of the subject, for example. The control unit 20 enables the subject to recognize unconscious thought information by causing the stimulation to be provided by the stimulation provision unit 30 to the predetermined region in the brain of the subject. For example, the stimulation provision unit 30 is capable of activating the predetermined region in the brain of the subject by emitting an electromagnetic wave signal to the predetermined region. In this case, the brain of the subject is sectioned by a three-dimensional matrix composed of voxels of a few millimeters or less, for example, and electromagnetic waves are emitted per voxel. The stimulation provision unit 30 is capable of emitting the electromagnetic waves on the basis of stimulation image information indicating electromagnetic waves of what intensity are to be emitted to which voxels in the three-dimensional matrix. As to what kind of thought information is recalled when electromagnetic waves of what intensity are emitted to which voxels of the brain of the subject, correspondence therebetween may be found by experiments beforehand.

The stimulation provision unit 30 may be a device that stimulates the five senses of the subject. In this case, for example, a device used as the stimulation provision unit 30 to stimulate the sight of the subject may be, for example, a display device, such as a monitor or a head-mounted display, or a light emission device that emits an optical signal. Furthermore, for example, a device used as the stimulation provision unit 30 to stimulate the hearing of the subject may be, for example, a sound playback device, such as a speaker. Furthermore, for example, a device used as the stimulation provision unit 30 to stimulate the touch of the subject may be, for example, a vibration device. Furthermore, for example, a device used as the stimulation provision unit 30 to stimulate the smell of the subject may be a device capable of spraying various flavor components, such as fragrances. Furthermore, for example, a device used as the stimulation provision unit 30 to stimulate the taste of the subject may be a device capable of supplying various taste components, such as a sweet taste, a hot taste, a salty taste, and a sour taste.

The storage unit 22 may store, for example, a pattern of information on conscious thought of the subject, a pattern of thought information at a point in time of a judgment, and a pattern of unconscious thought information. In this case, the storage unit 22 may store the content of the judgment by the subject, the pattern of the information on the conscious thought, the pattern of the thought information at the point in time of the judgment, and the pattern of the unconscious thought information, in association with one another. Furthermore, the storage unit 22 may store the correspondence with respect to what kind of thought information is recalled when electromagnetic waves of what intensity are emitted to which voxels of the brain of the subject.

Furthermore, the storage unit 22 stores an intracerebral information recognition program that causes a computer to execute: a process of detecting intracerebral information on a subject; and a process of causing, in a case where unconscious thought information generated prior to a point in time of a judgment made consciously upon the subject making the judgment is included in a result of detection of the intracerebral information, the unconscious thought information to be recognized at a point in time prior to the point in time of the judgment.

An intracerebral information recognition method using the intracerebral information recognition device 100 configured as described above will be described next. Firstly, a necessary device is attached to a subject to enable the detection unit 10 to detect intracerebral information on the subject. The detection unit 10 detects the intracerebral information on the subject and transmits a detection result to the control unit 20.

In a case where the subject makes a judgment and thinks unconsciously prior to a point in time of the judgment made consciously in this state, the detection result at the detection unit 10 includes unconscious thought information prior to the point in time of the judgment made consciously. The processing unit 21 determines whether or not the intracerebral information detected by the detection unit 10 includes unconscious thought information on the subject. For example, in a case where a pattern of unconscious thought information on the subject is stored in the storage unit 22 beforehand, the processing unit 21 is able to determine whether or not the pattern of the unconscious thought information stored in the storage unit 22 and a pattern of the intracerebral information from the detection result agree with each other or are similar to each other, by processing, such as pattern matching. In a case where the processing unit 21 has determined that these patterns agree with each other or are similar to each other, for example, the processing unit 21 is able to determine that the detection result from the detection unit 10 includes unconscious thought information.

In a case where the processing unit 21 has determined that the detection result from the detection unit 10 includes unconscious thought information, the processing unit 21 determines a form of a stimulation to be provided to the subject. For example, in a case where a configuration for providing the stimulation by emission of electromagnetic waves to a predetermined region in the brain of the subject is adopted, the correspondence with respect to what kind of thought information is recalled when electromagnetic waves of what intensity are emitted to which voxels of the brain of the subject is stored in the storage unit 22 beforehand. On the basis of the correspondence stored in the storage unit 22, the processing unit 21 determines the form of the stimulation so that electromagnetic waves are emitted to an appropriate predetermined region for unconscious thought information to be recognized.

In the following description, it is assumed that the form of the stimulation is determined at a point in time t3 prior to the point in time t2 of the judgment, as illustrated in FIG. 3. In this case, at the point in time t3 of the determination of the form of the stimulation, the processing unit 21 determines whether or not the point in time t3 is prior to the point in time t2 of the judgment made consciously by the subject. For example, in a case where a pattern of information on conscious thought of the subject or a pattern of thought information at a point in time of a judgment by the subject is stored in the storage unit 22 beforehand, the processing unit 21 is able to determine whether or not the pattern of the information on the conscious thought or the pattern of the thought information at the point in time of the judgment stored in the storage unit 22 is detected from a pattern of intracerebral information from a detection result, by processing, such as pattern matching. In a case where the pattern of the information on the conscious thought or the pattern of the thought information at the point in time of the judgment is not detected, the processing unit 21 is able to determine that the point in time t3 is prior to the point in time of the judgment.

In a case where the processing unit 21 has determined that the point in time t3 of the determination of the form of the stimulation is prior to the point in time t2 of the judgment, the processing unit 21 controls the stimulation provision unit 30 such that electromagnetic waves are emitted in the determined form, that is, to a predetermined region in the brain of the subject. On the basis of the control by the processing unit 21, the stimulation provision unit 30 emits electromagnetic waves to the predetermined region in the brain of the subject. By receiving the electromagnetic waves emitted to the predetermined region in the brain, a sense corresponding to unconscious thought information is recalled and the subject is able to recognize the unconscious thought information. As a result, the subject is able to recognize the unconscious thought information at a point in time t4 prior to the point in time t2 of the judgment.

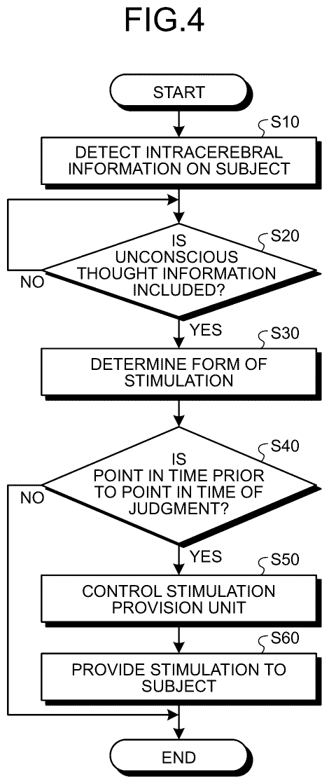

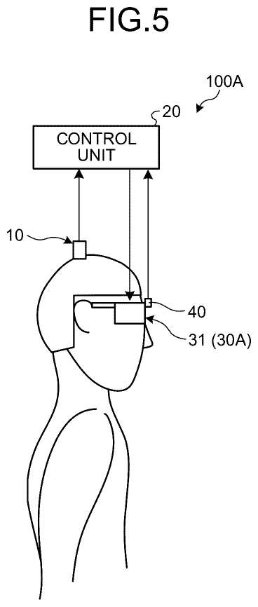

FIG. 4 is a diagram schematically illustrating another example of the form of providing a stimulation to a subject. As illustrated in FIG. 5, a stimulation is able to be provided by stimulation of the sight of the subject, for example. In FIG. 5, the stimulation provision unit 30 has a display unit 31. The display unit 31 displays information, such as an image. The processing unit 21 is capable of causing an appropriate image to be displayed on the display unit 31 for unconscious thought information to be recognized by the subject. Such an image may be stored in the storage unit 22 in association with the content of a judgment made by the subject, for example.

Furthermore, on the basis of the unconscious thought information, the processing unit 21 may predict a phenomenon to be subjected to a judgment by the subject. In this case, correspondence between patterns of unconscious thought information and phenomena to be subjected to judgments may be stored as a trained model in the storage unit 22 beforehand. The trained model is trained to output an optimum phenomenon corresponding to unconscious thought information in a case where a pattern of the unconscious thought information is input to the trained model. The processing unit 21 is then able to predict a phenomenon on the basis of an output result in a case where a pattern of unconscious thought information included in a detection result from the detection unit 10 is input to the trained model. The processing unit 21 selects an image corresponding to the predicted phenomenon and causes the image to be displayed on the display unit 31 for recognition by the subject.

FIG. 4 is a flowchart illustrating an example of an intracerebral information recognition method according to the embodiment. As illustrated in FIG. 4, intracerebral information on a subject is detected by the detection unit 10 (Step S10). The detection unit 10 transmits a detection result to the control unit 20.

At the control unit 20, the processing unit 21 determines whether or not the intracerebral information detected by the detection unit 10 includes unconscious thought information on the subject (Step S20). In a case where the processing unit 21 has determined that the detection result from the detection unit 10 does not include unconscious thought information (Step S20: No), the processing unit 21 repeats the processing at Step S20. On the contrary, in a case where the processing unit 21 has determined that the detection result from the detection unit 10 includes unconscious thought information on the subject (Step S20: Yes), the processing unit 21 determines a form of a stimulation to be provided to the subject (Step S30).

The processing unit 21 determines whether or not a point time of the determination of the form of the stimulation is prior to a point in time of a judgment made consciously by the subject (Step S40). In a case where the processing unit 21 has determined that the point in time of the determination of the form of the stimulation is prior to the point in time of the judgment (Step S40: Yes), the processing unit 21 controls the stimulation provision unit 30 such that the subject is stimulated in the form determined (Step S50). On the basis of the control by the processing unit 21, the stimulation provision unit 30 provides a stimulation to the subject (Step S60) and ends processing. In a case where the processing unit 21 has determined at Step S40 that the point in time of the determination of the form of the stimulation is not prior to the point in time of the judgment (Step S40: No), the processing unit 21 skips the processing at Step S50 and Step S60 and ends processing.

As described above, the intracerebral information recognition device 100 according to the embodiment includes: the detection unit 10 that detects intracerebral information on a subject; the stimulation provision unit 30 that provides a stimulation to the subject; and a control unit 20 that controls, in a case where unconscious thought information generated prior to a point in time of a judgment made consciously upon the subject making the judgment is included in a result of the detection of the intracerebral information, the stimulation provision unit 30 such that a stimulation for causing the unconscious thought information to be recognized is provided to the subject at a point in time prior to the point in time of the judgment.

The intracerebral information recognition method according to the embodiment includes: detecting intracerebral information on a subject; and causing, in a case where unconscious thought information generated prior to a point in time of a judgment made consciously upon the subject making the judgment is included in a result of the detection of the intracerebral information, the unconscious thought information to be recognized at a point in time prior to the point in time of the judgment.

The intracerebral information recognition program according to the embodiment causes a computer to execute: a process of detecting intracerebral information on a subject; and a process of causing, in a case where unconscious thought information generated prior to a point in time of a judgment made consciously upon the subject making the judgment is included in a result of the detection of the intracerebral information, the unconscious thought information to be recognized at a point in time prior to the point in time of the judgment.

This configuration enables, in a case where unconscious thought information is generated prior to a point in time of a judgment made consciously upon a subject making the judgment, the subject to appropriately recognize the unconscious thought information at the point in time prior to the point in time of the judgment. The unconscious thought information is thereby able to be made useful to the judgment by the subject.

The stimulation provision unit 30 in the intracerebral information recognition device 100 according to the embodiment provides a stimulation that causes predetermined sensation at a predetermined region in the brain of a subject by emitting electromagnetic waves to the predetermined region. This configuration enables the subject to appropriately recognize unconscious thought information.

The stimulation provision unit 30 in the intracerebral information recognition device 100 according to the embodiment provides a stimulation to at least one of the five senses of a subject. This configuration enables the subject to appropriately recognize unconscious thought information.

The technical scope of the present disclosure is not to be limited to the embodiment described above and modifications may be made as appropriate without departing from the gist of the present disclosure. FIG. 5 is a schematic diagram illustrating another example of the intracerebral information recognition device according to the embodiment. An intracerebral information recognition device 100A illustrated in FIG. 5 includes a detection unit 10, a control unit 20, and a stimulation provision unit 30A. The detection unit 10 and the control unit 20 have hardware configurations similar to those of the intracerebral information recognition device 100 described above. The intracerebral information recognition device 100A has, as the stimulation provision unit 30A, a display unit 31 that displays information on an image, for example. A head-mounted display will be described hereinafter as an example of the display unit 31 but the display unit 31 is not limited to this example. The control unit 20 obtains a video captured by an imaging unit 40. The imaging unit 40 is provided integrally with the display unit 31, for example, and captures a video of surroundings of a subject. The video captured by the imaging unit 40 is obtained as information on the surroundings of the subject (environment information).

The intracerebral information recognition device 100A illustrated in FIG. 5 enables a stimulation to be provided by stimulation of the sight of the subject, for example. A processing unit 21 in the intracerebral information recognition device 100A is capable of causing an appropriate image to be displayed on the display unit 31 for causing the subject to recognize unconscious thought information. Such an image may be stored in a storage unit 22 in association with the content of a judgment made by the subject, for example.

Furthermore, the processing unit 21 may predict a phenomenon to be subjected to a judgment by the subject on the basis of unconscious thought information included in intracerebral information detected by the detection unit 10. In this case, correspondence between patterns of unconscious thought information and phenomena to be subjected to judgments are stored as a trained model in the storage unit 22 beforehand. This trained model is trained beforehand to output an optimum phenomenon corresponding to unconscious thought information in a case where a pattern of the unconscious thought information is input to the trained model. The processing unit 21 is thereby able to predict a phenomenon on the basis of an output result in a case where a pattern of unconscious thought information included in a detection result from the detection unit 10 is input to the trained model. The processing unit 21 selects an image corresponding to the predicted phenomenon and causes the image to be displayed on the display unit 31 for recognition by the subject. As described above, in determining a form of a stimulation, the processing unit 21 is able to determine the form of the stimulation on the basis of a prediction result.

In this case, the processing unit 21 may select an image on the basis of the phenomenon predicted and environment information captured by the imaging unit 40. For example, it is assumed that a subject is walking on a road toward an intersection with poor visibility, an automobile is traveling on a road toward the intersection, the road intersecting the road where the subject is walking on, and the subject has not consciously recognized this automobile. It is also assumed that the imaging unit 40 is capturing a region in front of the subject, that is, a view including the intersection. In a case where unconscious thought information is detected in a detection result from the detection unit 10 in this situation, the processing unit 21 is able to predict a phenomenon on the basis of the unconscious thought information and a video captured by the imaging unit 40. For example, the processing unit 21 is able to predict, as the phenomenon, that the automobile is going to run out from somewhere hidden at the intersection captured in the video. In this case, information to warn the subject that the automobile is going to run out to the intersection may be displayed on the display unit 31, with the information superposed on the video of the intersection captured by the imaging unit 40.

Furthermore, the processing unit 21 may control the stimulation provision unit 30A on the basis of unconscious thought information included in intracerebral information detected by the detection unit 10 and environment information. In this case, the storage unit 22 stores a pattern of the unconscious thought information and a detection pattern for the environment information in association with each other. In a case where the processing unit 21 has determined that the intracerebral information includes the pattern of the unconscious thought information, the processing unit 21 determines whether or not the environment information includes the detection pattern. In a case where the processing unit 21 has determined that the environment information includes the detection pattern, the processing unit 21 is able to provide a stimulation by selecting an image or a sound for warning and causing the display unit 31 to display the image or outputting the sound by means of a speaker (not illustrated in the drawings).

For example, it is now assumed that a subject is walking on the side of a road and an automobile is traveling on that road. It is also assumed that the imaging unit 40 has been capturing a video of the road and the automobile, for example. The processing unit 21 determines whether or not intracerebral information includes a pattern of unconscious thought information recorded in the storage unit 22. In this case, for example, the pattern of the unconscious thought information is a pattern of unconscious thought information with respect to running out. In a case where the processing unit 21 has determined that the intracerebral information includes the pattern of the unconscious thought information for running out, the processing unit 21 determines whether or not environment information includes a detection pattern corresponding to the pattern of the unconscious thought information. The environment information herein is the video captured by the imaging unit 40. The corresponding detection pattern is, for example, a movement of a moving body, such as an approach of an automobile. In a case where the environment information includes the detection pattern corresponding to the pattern of the unconscious thought information, that is, in a case where the processing unit 21 has detected the approach of the automobile in the video captured, the processing unit 21 displays a warning to stop running out, on the display unit 31. In a case where the processing unit 21 has determined that the intracerebral information does not include the pattern of the unconscious thought information recorded in the storage unit 22, the processing unit 21 ends processing if the environment information does not include the detection pattern corresponding to the pattern of the unconscious thought information.

The processing unit 21 may select a pattern of unconscious thought information on the basis of environment information. For example, in a case where the environment information includes a road, the processing unit 21 may select a pattern of unconscious thought information for running out, and in a case where the environment information indicates that a subject is in a room, the processing unit 21 may select a pattern of unconscious thought information for walking, and the processing unit 21 may then determine whether or not the intracerebral information includes the selected pattern of the unconscious thought information.

As described above, the processing unit 21 in the intracerebral information recognition device 100A according to the embodiment provides a stimulation to a subject in a case where unconscious thought information generated prior to a point in time of a judgment made consciously upon the subject making the judgment is included in a result of detection of intracerebral information and a pattern corresponding to the unconscious thought information is included in environment information that is surrounding information on the subject. This configuration enables the subject to appropriately recognize the unconscious thought information.

The processing unit 21 may predict a phenomenon on the basis of environment information different from a video captured by the imaging unit 40. Examples of such environment information include a temperature around the subject, the weather, the time, or a sound collected around the subject. In a case where intracerebral information detected by the detection unit 10 includes other information different from unconscious thought information in a time period prior to a point in time of a judgment, the processing unit 21 may predict a phenomenon on the basis of that other information instead of environment information.

The present disclosure enables appropriate conscious recognition of information generated unconsciously upon a subject making a judgment.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An intracerebral information recognition device, comprising:
a detection unit that detects intracerebral information on a subject;
a stimulation provision unit that provides a stimulation to the subject; and
a control unit that controls, in a case where a result of detection of the intracerebral information includes unconscious thought information generated prior to a point in time of a judgment made consciously upon the subject making the judgment, the stimulation provision unit such that the stimulation is provided at a point in time prior to the point in time of the judgment.

2. The intracerebral information recognition device according to claim 1, wherein the stimulation provision unit provides a stimulation that causes predetermined sensation at a predetermined region in the brain of the subject by emitting electromagnetic waves to the predetermined region.

3. The intracerebral information recognition device according to claim 1, wherein the stimulation provision unit provides a stimulation to at least one of the five senses of the subject.

4. The intracerebral information recognition device according to claim 1, wherein the control unit provides the stimulation to the subject in a case where the unconscious thought information generated prior to the point in time of the judgment made consciously upon the subject making the judgment is included in the result of the detection of the intracerebral information and a pattern corresponding to the unconscious thought information is included in environment information that is surrounding information on the subject.

5. An intracerebral information recognition method, including:
detecting intracerebral information on a subject; and
causing, in a case where a result of detection of the intracerebral information includes unconscious thought information generated prior to a point in time of a judgment made consciously upon the subject making the judgment, the unconscious thought information to be recognized at a point in time prior to the point in time of the judgment.

6. A non-transitory computer-readable storage medium storing a program causing a computer to execute:
a process of detecting intracerebral information on a subject; and
a process of causing, in a case where a result of detection of the intracerebral information includes unconscious thought information generated prior to a point in time of a judgment made consciously upon the subject making the judgment, the unconscious thought information to be recognized at a point in time prior to the point in time of the judgment.
